(19) United States
(12) Patent Application Publication
(10) Pub. No.: US 2013/0016319 A1
(43) Pub. Date: Jan. 17, 2013

(54) RETINAL IMAGING SYSTEMS WITH IMPROVED RESOLUTION

(71) Applicants: Brian Vohnsen, The Grange Stillorgan (IE); Diego Jose Rativa Milan, Kings Court Smithfield (IE)

(72) Inventors: Brian Vohnsen, The Grange Stillorgan (IE); Diego Jose Rativa Milan, Kings Court Smithfield (IE)

(73) Assignee: University College Dublin, National University of Ireland, Dublin, Dublin (IE)

(21) Appl. No.: 13/518,794

(22) PCT Filed: Dec. 23, 2010
(86) PCT No.: PCT/EP2010/070716

§ 371 Date: Aug. 21, 2012

(30) Foreign Application Priority Data
Dec. 23, 2009 (IE) 2009/0963
Jan. 8, 2010 (IE) S2010/0003

(51) Int. Cl. A61B 3/12; A61B 3/14

(52) U.S. Cl. 351/206; 351/246

(57) ABSTRACT

A scanning ophthalmoscope focuses coherent light to a target area in which a subject's eye is located. One or more scanning stages direct the light in a scanning pattern within the target area, and an imaging detector receives a reflected light signal returned following retinal reflection in the subject's eye. Adaptive optics compensate for aberrations in the wavefront. The light is provided as an annular beam at a plane which is conjugate with the pupil of a subject whose eye is located in the target area, whereby the annular beam is focused from an annulus at the pupil of the eye to a spot at the fundus of the eye. The spot size resulting from using an annular beam in this way is significantly reduced providing enhanced resolution.

### TECHNICAL FIELD

This invention relates to retinal imaging systems such as scanning ophthalmoscopes and optical coherence tomography (OCT) systems.

### BACKGROUND ART

Scanning ophthalmoscopes provide real-time imaging of the living eye of a subject. Conventional ophthalmoscopes employ flood illumination which results in a lower contrast in the final image than using a scanning source (typically a laser source) and a confocal pinhole.

Diseases and damage to the eye most frequently occurs at the front elements (cornea and lens) or the rear elements (retina and optic nerve). The former elements are easier to access, visualise and surgically treat than the latter. When damage occurs at the retina or optic nerve it is often untreatable. Accordingly early diagnosis is important. Currently, however, imaging in vivo of the fovea (central part of the retina where the photoreceptors are most tightly packed) is not possible with a resolution high enough to see individual photoreceptors.

Roorda et al. have disclosed a scanning laser ophthalmoscope which uses adaptive optics to correct for higher order aberrations of the human eye (Roorda, A., Romero-Borja, F., Donnelly III, W. J., Queener, H., Hebert, T. J., Campbell, M. C. W. “Adaptive Optics Scanning Laser Ophthalmoscopy” Opt. Express 10(9) 405-412 (2002)).

The system disclosed by Roorda et al. employs a diode laser coupled into a fibre providing a point source, collimated by a lens. A series of mirror telescopes relays light via a deformable mirror to horizontal and vertical scanning stages, and finally to the eye. A wavefront sensor sees light reflected from the retina and measures any aberration, and a deformable mirror is driven by the wavefront sensor to correct aberrations. The light from the retina is de-scanned and focused onto a confocal pinhole before being detected with a photomultiplier tube and fed to an analog frame grabbing board.

A drawback with this system is that the resolution is limited, even with the introduction of adaptive optics and a larger eye pupil. With near-infrared light, the focused spot size is limited to approximately four microns and is thus too large to probe individual central fovea photoreceptors or small rod photoreceptors. One solution is to use a shorter wavelength of light, but this runs the risk of damaging the front elements of the eye.

Optical coherence tomography has also been used in retinal and other biomedical imaging. In OCT systems, an interferometer is used to image reflected light from a tissue such as the retina. OCT systems may operate in the time domain or frequency domain. OCT systems typically give very high depth resolution, but as with scanning ophtalmoscopes, lateral resolution of detected features is a limiting factor in the imaging process.

### DISCLOSURE OF THE INVENTION

The invention provides a scanning ophthalmoscope comprising:
(a) a coherent light source
(b) focusing optics to direct light from said light source along a path to a target area in which a subject's eye is to be located;
(c) one or more scanning stages located along said path for directing said light in a scanning pattern within said target area;
(d) an imaging detector for receiving a reflected light signal returned along said path following retinal reflection in a subject's eye; and
(e) an adaptive optics system comprising a detector for detecting aberrations in the wavefront of light caused as the light travels along the path to and from the retina and a wavefront correction system located along said path for compensating for said aberrations;
characterised by
(f) means for providing said light directed along said path as an annular beam at a plane which is conjugate with the pupil of a subject whose eye is located in said target area.

The use of an annular beam at the entrance pupil allows the light to be focused to a central spot which is smaller than the diffraction limit of a fully illuminated pupil.

Thus, the beam of light which is focused by the eye onto a spot on the retina is conical and tapers to a point on the retina, as with conventional systems, but with the difference that the “cone” is effectively hollow as opposed to solid. In other words, the path along the central axis of the “cone” and immediately surrounding this central axis is not illuminated. This means that all ray paths reaching the retina are constrained to impinge on the retinal surface at an oblique angle, i.e. no rays strike the retina on a path which is normal or perpendicular to the retina. In turn this means that the definition of the interference pattern resulting from interaction of the rays is more highly defined with a central peak or point having a far smaller lateral spread, surrounded by higher definition rings where higher order interference fringes are formed.

The narrower focus of the scanning light spot is accompanied by increased ringing of light away from the central bright spot. The imaging system can exclude such rings from the final image due to the spatial separation from the central bright point. In contrast, systems which do not exclude light rays from impinging on the retinal along a normal or perpendicular path give rise to a far less sharply defined interference pattern, with a brighter central spot, but where this spot is spread over a greater lateral area, and usually without any distinct separation from less bright higher order interference fringes.

The means for providing the light as an annular beam can be integral with the coherent light source, whereby the light source outputs an annular beam. One example of this is to use a laser operating in the so-called doughnut mode or TEM01 mode.

Alternatively, the means for providing the light as an annular beam comprises a beam shaping system positioned to receive light from the light source and to shape the light into an annular beam at an output of the system, the output being located in a plane conjugate with the pupil of a subject whose eye is located in the target area.

Preferably, the beam shaping system comprises an annular filter, an axicone system, a phase plate or a hologram.

An annular filter or an axicone telescope are the preferred options. The former is the simplest, but the latter provides less attenuation of the light.

Preferably, the detector of the adaptive optics system is positioned to receive a first portion of reflected light from the retina and wherein the imaging detector is positioned to receive a second portion of reflected light from the retina, and further comprising a beam splitter for dividing the reflected light into the first and second portions.

Further, preferably, the wavefront correction system is located along the path between the beam splitter and the target area.

The ophthalmoscope may further comprise a confocal pinhole sized to eliminate interference rings reflected from a subject's retina around a central focal point.

The invention also provides an optical coherence tomography system in which a low coherence source is employed and is imaged onto a target area in which a subject's eye is to be located, characterised in that the illumination arm of the system comprises means for providing light from the low coherence source as an annular beam at a plane which is conjugate with the pupil of a subject whose eye is located in said target area

Using such an illumination system, and in a corresponding manner to the illumination portion of the FIG. 1 ophthalmoscope, a high transverse resolution will be obtained simultaneously with the high depth resolution that is characteristic of the OCT.

### BRIEF DESCRIPTION OF THE DRAWINGS

FIG. 1 is a schematic layout of the optical components of a scanning ophthalmoscope;

FIG. 2 is a light ray diagram showing the focusing of a conventional scanning ophthalmoscope on a surface such as a retina;

FIG. 3 is a light ray diagram, corresponding to that of FIG. 2, but showing the focusing of the scanning ophthalmoscope of FIG. 1 on a surface such as a retina; and

FIG. 4 is a dual plot of normalised intensity against diameter for the light patterns produced by the systems of FIGS. 2 and 3 respectively.

### DETAILED DESCRIPTION OF PREFERRED EMBODIMENTS

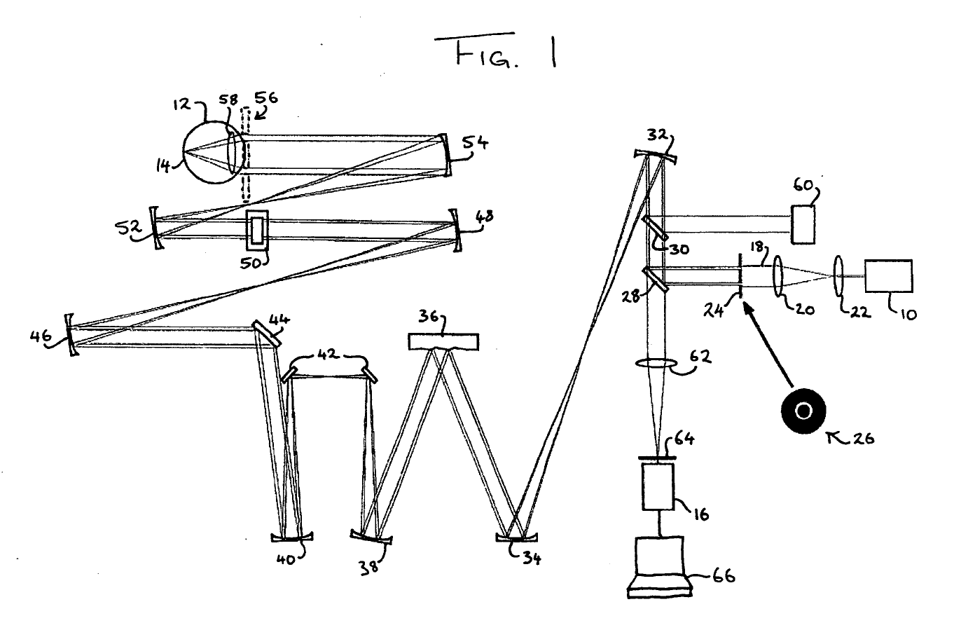

In FIG. 1 there is shown a scanning ophthalmoscope which uses a coherent light source 10 directed to the eye 12 of a human or animal subject. Incident light reflected from the retina 14 of the eye is collected at a light detector 16.

The coherent light source 10 takes the form of a near-infrared laser 785 nm wavelength, 100 mW power (Newport, LQC785-100C). Light from the laser is collimated and spread out into a parallel beam 18 by a pair of lenses 20,22. The beam 18 in incident on an annular filter 24 which is shown in a front view at 26. Thus, the light emerging from annular filter 24 is an annular beam of light. A first beam splitter 28 directs a portion of the light upwards and through a second beam splitter 30 to a first telescope comprising a pair of concave mirrors 32,24. (It is to be understood that directional terms such as “upwards” are used for convenience in describing the drawing, and the orientation of the system is immaterial.) Each mirror pair is a 4f system that images the eye pupil plane 56 onto conjugate planes containing a spatial wavefront modulator such as a deformable mirror 36, the wavefront sensor 60, and the annular filter 24. This ensures that all corrections and changes are performed in a plane conjugate with the eye pupil (otherwise there would be diffractive propagation effects).

A wavefront modulator 36 modulates the beam of light on both the forward and return passes. The wavefront modulator is a deformable mirror membrane (Boston Micromachines Corporation) with 140 actuators and a total 3.5 micron stroke. The modulator corrects for aberrations introduced by the eye and by the optical system itself.

The annular beam from the wavefront modulator is directed to a further telescope consisting of a pair of mirrors 38,40 and a Badal system 42. The Badal system has been included to correct for defocus of the subject's eye and bring the aberrations within the operative range of the wavefront modulator used. After mirror 40, the light is directed to a horizontal resonant scanner 44 from GSI Lumonics operating at a scan rate of 12.0 kHz, and from there via a further telescope 46,48 to a vertical scanner from GSI Lumonics operating at a rate of approximately 23.5 Hz identical to the frame rate. The horizontal and vertical scanners 44,50 operate together to sweep the light in a raster pattern across the retina. The horizontal and vertical scanners work together like a TV raster scan, “painting” the scan pattern line by line, i.e. for a 512×512 picture, the horizontal scan rate is continuous, while the vertical scan operates to deflect the beam stepwise after each forward and backward horizontal scan, so that 512 small deflections of the vertical scanner fill out the full scan area. The system shown scans a retinal area of 2×2 visual (this being adjustable). Image pixels are only recorded in the forward scan direction of the resonant scanner but can easily be recorded also in the return path.

A final telescope consisting of a pair of mirrors 52,54 directs the scanning annular beam into the subject's eye 12. Each of the mirrors 32,34,38,40,46,48,54 has a focal length of 200 mm, while the mirror 52 forming part of the final telescope has a focal length of 100 mm, such that the diameter of the annular beam is increased from 3 mm (this being the diameter of the annular aperture in filter 26) to 6 mm (this being the dilated pupil diameter of an adult eye). A real image of the annular aperture is shown in dotted outline at 56. The image of the annular aperture 56 is located in a plane conjugate with the wavefront modulator 36, the wavefront sensor 60, and the annular filter 26.

The cornea and lens 58 of the eye 12 focuses the annular beam of light into a “hollow cone” of light whose apex is at the fovea of the retina 14. (The ophthalmoscope can of course be manipulated to focus on other parts of the retina.)

Reflected light from the fovea passes back through the optical system of the ophthalmoscope in a reverse path until it meets beam splitter 30 where a first portion of the reflected light is directed to the Hartmann-Shack wavefront detector 60 (Thorlabs WFS 150C) and a second portion is directed towards beam splitter 28. The first portion of light is used by the Hartmann-Shack detector to control the adaptive mirror 36 in order to maintain as closely as possible a planar wavefront. The second portion of light is partly reflected by beam splitter 28, but the majority passes through beam splitter 28 to a focusing lens 62 onto a confocal pinhole 64, the function of which will be described below. Light passing through the confocal pinhole reaches the detector 16 which is a photomultiplier tube (Sens-Tech) or avalanche photodiode.

Image acquisition is done at a frame rate of 24 Hz set by the resident galvanometric scanner of the system (12 kHz). The detector builds up a 512×512 pixel image which is sent to a PC 66 where appropriate video hardware and software produces a moving image for display, recordal and analysis using conventional video techniques. The detector takes a series of 262,144 snapshots, each of which is one pixel of the final 512×512 image.

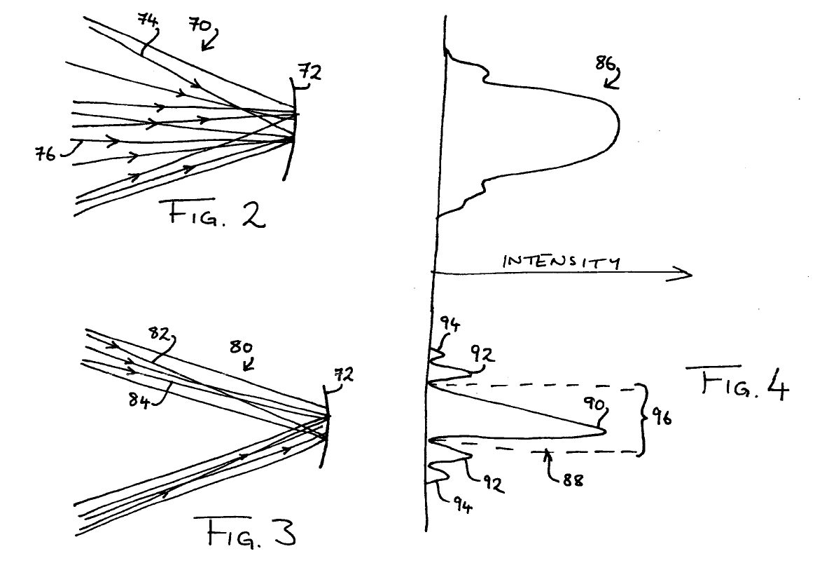

Referring now to FIGS. 2 and 3, the effect of the annular aperture 24 in the system of FIG. 1 can be illustrated. FIG. 2 shows the effect of focusing a circular beam of light into a cone, indicated generally at 70, which is focused as tightly as possible on a surface 72, such as a retina. The highly ordered coherent beam ensures that all rays within the illumination cone intersect the retina in an ordered arrangement. Constructive interference on axis ensures that the net result is a normally incident wavefront on the retina or similar.

In contrast, FIG. 3 shows how an annular beam of light is focused into a “hollow cone” onto a similar sized area. In contrast to FIG. 2, it can be seen that all of the light is confined to the “surface” of the hollow cone of light 80. All rays are constrained to follow a path (due to the annular aperture) which leads them to impinge on the surface 72 at a similar oblique angle. The approximate half-cone-angle for the rays from an annular 6 mm pupil is 8 degrees.

The effect of these different approaches, as illustrated in FIGS. 2 and 3, is shown in FIG. 4. FIG. 4 shows a graph of intensity of light, as seen at the surface 72, i.e. the intensity of the spot of light generated due to interference of the incident rays. The upper plot 86 is illustrative of the interference pattern resulting from the approach of FIG. 2, where light rays arriving at a wide range of angles combine to produce a poorly defined interference pattern which is spread out over a considerable area (in FIG. 4, the horizontal axis represents distance along the surface 72 while the vertical axis represents intensity of light). Thus, it can be seen that in general, interference effects mean that the spot of light produced on surface 72 is far greater than the geometrical projection of the cone apex onto the surface, simply as a result of the interference pattern “smearing” the light spot.

In contrast, the continuous line plot 88 in FIG. 4 shows a much better defined interference pattern, with a zero-order peak 90 localised and distinguishable from first order peaks 92 and higher order peaks 94.

It should be noted that the plots 86,88 have been normalised along the intensity scale. In actuality, the spot produced in FIG. 2 will be much brighter than that produced in FIG. 3, all else being equal, due to the occlusion of a portion of the light by the annular aperture and the accompanying reduction in intensity.

As indicated by bracket 96, it is possible to filter out the first and higher order peaks 92,94 from the reflected intensity pattern 88, so that just the zero-order peak 90 is observed. The reflected image from the retina of intensity pattern 88 shows a brighter central spot surrounded by fainter rings representing the higher order fringes. The confocal pinhole 64 in FIG. 1 is positioned and sized to allow through the zero-order peak (bright central spot) and to intercept the surrounding rings, so that only the bright spot is imaged, this having a spatial extent which is of the same order as the width of the photoreceptors of the fovea.

It has been experimentally verified that the diameter of the bright spot produced by an annular aperture as opposed to a full unobstructed pupil (i.e. produced by the approach of FIG. 3 as opposed to FIG. 2), results in an approximately three-fold reduction in the area of the light spot produced on the retina.

The ideal coupling of incident light to an individual photoreceptor is obtained when the width of the incident beam is perfectly matched to the width of the photoreceptor. When the size of the spot at the focus is larger, or even smaller, the amount of coupled light is reduced and in consequence the reflected light power is less. Also, when the spot is wider than the individual photoreceptors it will interpret more than one at a time, preventing them from being resolved in the recorded image, which is typically the case for commercially available current systems.

The system of FIG. 1 can be modified in various ways, in particular by using beam transforming elements other than a simple annular filter. A telescopic set of axicons can provide a similar annular beam of light, which will allow a less powerful laser source to be used as it transmits light more efficiently.

Furthermore, to provide optimal interference conditions for the incident light, pre-compensation for corneal birefringence will lead to a linear polarisation state of the light reaching the retina and thus an optimally reduced light spot. At present, wavefront aberrations across the entire pupil are corrected and kept at a minimum in real time with the adaptive optics system consisting of the deformable membrane mirror and the Hartmann-Shack wavefront sensor. Since optimal focusing is achieved with a narrow annular beam of light, it is also possible to control the adaptive optics only in the annular ring, whereby the resources of the adaptive optics can be concentrated on the most critical parts of the wavefront.

The invention is not limited to the embodiment(s) described herein but can be amended or modified without departing from the scope of the present invention.

1. A scanning ophthalmoscope comprising:
(a) a coherent light source
(b) focusing optics to direct light from said light source along a path to a target area in which a subject's eye is to be located;
(c) one or more scanning stages located along said path for directing said light in a scanning pattern within said target area;
(d) an imaging detector for receiving a reflected light signal returned along said path following retinal reflection in a subject's eye;
(e) an adaptive optics system comprising a detector for detecting aberrations in the wavefront of light caused as the light travels along the path to and from the retina and a wavefront correction system located along said path for compensating for said aberrations; and
(f) means for providing said light directed along said path as an annular beam at a plane which is conjugate with the pupil of a subject whose eye is located in said target area, whereby said annular beam is focused from an annulus at the pupil of the eye to a spot at the fundus of the eye.

2. A scanning ophthalmoscope as claimed in claim 1, wherein said means for providing said light as an annular beam is integral with said coherent light source, whereby said light source outputs an annular beam.

3. A scanning ophthalmoscope as claimed in claim 1, wherein said means for providing said light as an annular beam comprises a beam shaping system positioned to receive light from said light source and to shape the light into an annular beam at an output of the system, the output being located in a plane conjugate with the pupil of a subject whose eye is located in said target area.

4. A scanning ophthalmoscope as claimed in claim 3, wherein the beam shaping system comprises an annular filter, an axicone, a phase plate or a hologram.

5. A scanning ophthalmoscope as claimed in claim 1, wherein the detector of the adaptive optics system is positioned to receive a first portion of reflected light from the retina and wherein the imaging detector is positioned to receive a second portion of reflected light from the retina, and further comprising a beam splitter for dividing said reflected light into the first and second portions.

6. A scanning ophthalmoscope as claimed in claim 5 wherein the wavefront correction system is located along the path between the beam splitter and the target area.

7. A scanning ophthalmoscope as claimed in claim 1, further comprising a confocal pinhole sized to eliminate interference rings reflected from a subject's retina around a central focal point.

8. A scanning ophthalmoscope as claimed in claim 1, wherein the beam of light, which is haract by the eye onto a spot on the fundus of the eye, is conical and tapers to said spot on the retina, the cone of light being hollow.

9. A scanning ophthalmoscope as claimed in claim 1, wherein no illumination reaches the fundus along a path normal to the fundus at said spot.

10. A method of imaging the fundus of an eye, comprising the steps of:
(a) focusing coherent light from along a path to a target area in which a subject's eye is to be located
(b) directing said light in a scanning pattern within said target area;
(c) receiving a reflected light signal returned along said path following retinal reflection in a subject's eye;
(d) compensating for aberrations in the wavefront of light caused as the light travels along the path to and from the retina with an adaptive optics system; and
(e) providing said light directed along said path as an annular beam at a plane which is conjugate with the pupil of a subject whose eye is located in said target area, whereby said annular beam is focused from an annulus at the pupil of the eye to a spot at the fundus of the eye.

11. An optical coherence tomography system in which a low coherence source is employed and is imaged onto a target area in which a subject's eye is to be located, characterised in that the illumination arm of the system comprises means for providing light from the low coherence source as an annular beam at a plane which is conjugate with the pupil of a subject whose eye is located in said target area.
